(19) United States
(12) Patent Application Publication
(10) Pub. No.: US 2026/0108576 A1
(43) Pub. Date: Apr. 23, 2026

(54) COMPOSITION AGAINST ECTOPARASITES

(71) Applicant: BIODEVAS LABORATOIRES, Savigne-l'Evêque (FR)

(72) Inventors: François BLUA, Le Mans (FR); Jean Louis BLUA, Savigne-l'Evêque (FR)

(73) Assignee: BIODEVAS LABORATOIRES, Savigne-l'Evêque (FR)

(21) Appl. No.: 19/428,445

(22) Filed: Dec. 22, 2025

Related U.S. Application Data
(62) Division of application No. 17/607,727.

(30) Foreign Application Priority Data
Apr. 30, 2019 (FR) FR1904596

(51) Int. Cl. A61K 36/53; A23K 10/30; A23K 50/42; A61K 36/282; A61K 36/48; A61K 36/899; A61P 33/14

(52) U.S. Cl. CPC A61K 36/53; A23K 10/30; A23K 50/42; A61K 36/282; A61K 36/48; A61K 36/899; A61P 33/14

(57) ABSTRACT

A composition including a mixture of extracts of aromatic plants, and in particular extracts of lemon balm, thyme, rosemary, wormwood and lemongrass, veterinary or pharmaceutical compositions including the composition and a food for non-human animals including the composition. Also, a method for preparing the composition and a method for the prevention or treatment of flea infestation that include the administration of the composition to a domestic mammal.

### FIELD

The invention relates to an anti-ectoparasitic composition, in particular a composition intended to protect against and/or eliminate external parasites of pets.

### BACKGROUND

In all epidemiological studies, flea infestation (pulicosis) is undoubtedly the leading cause of annoyance in domestic carnivores. Fleas are biting insects whose adults live on mammals and birds. The species of fleas most commonly found in domestic carnivores in France is Ctenocephalides felis. More rarely, it is possible to find fleas from hedgehogs, birds, rabbits and sometimes even human fleas on cats and dogs.

Adult fleas can only live on an animal, while eggs and larvae develop in the external environment.

Once on their host, the adults feed on blood by piercing the skin of the animal that hosts them. Their life expectancy is in theory several months, but they are often eliminated after one to three weeks when the dog or cat grooms or bites itself.

The life cycle of fleas is dependent on environmental conditions. When the conditions are ideal (humidity, heat, nearby hosts), all the stages of development can be accomplished in less than 2 weeks! A single female flea can thus produce tens of thousands of new adults in a month. The number of fleas on animals and in particular in the environment can very quickly become considerable.

Since heat is necessary for the survival of flea larvae, they can thrive outdoors from spring to fall, but not in winter. However, heating houses and apartments allows them to grow indoors throughout the year.

It is important to realize that flea contamination of an animal is mainly from pre-emerging adults present in the environment, and that this contamination can come from outside (except in winter) as well as from inside the house. Contamination by the passage of an adult flea from one animal to another is certainly possible, but ultimately rather rare. It requires close contact between the two animals.

Flea infestation is known as pulicosis. The presence of fleas on an animal has many disadvantages for it:
Pain felt by the animal during the bite,
Itching,
Blood loss in case of over-infestation, and
The risk of disease transmission. Fleas can transmit bacterial diseases to cats, including cat scratch disease, with which cats can then infect humans. Fleas in dogs and cats are also vectors of a digestive worm known as Dipylidium caninum.

Fleas in pets can also occasionally bite humans, causing pimples and itching. This happens in particular when there is a large number of young adults in the environment. Improperly referred to as “floor fleas,” they are in fact pre-emerging adult dog or cat fleas, which hatch suddenly and in large numbers when they sense the vibrations produced by the footsteps of a person entering a room. These young, hungry fleas then bite any human who passes within their reach.

There are many products for treating fleas in pets. They are differentiated by their composition, their form and their duration of action.

Most are veterinary drugs that may or may not require a prescription, with chemical molecules of which the galenic formulation may present a toxic risk for young children. This is particularly the case with anti-parasitic collars and spot-on products commonly offered by veterinarians, which paradoxically no longer require a prescription and are available free of charge in specialist stores.

Other galenic formulations, such as tablets, present less risk but, with an effective duration generally of one month, these molecules place great strain on the animal's liver.

On the other hand, there is a desire on the part of owners to have simple preventive treatments that are not without side effects for the animals.

The new regulations concerning sensory additives for veterinary use allow a return to the use of aromatic plant extracts, which makes it possible to compensate for the use of chemical compositions in certain cases and achieve the desired objectives. This is the case in digital dermatitis, as proposed by FR3026643.

However, to date there is no such composition for protecting against fleas.

### SUMMARY

As such, the aim of the invention is to remedy these shortcomings of the prior art.

One of the aims of the invention is to provide a natural and effective means of preventing and/or eliminating fleas in farmed animals, without the use of chemicals.

Another aim of the invention is to provide a simple production method for this means of eliminating fleas.

As such, the invention relates to a composition comprising, or consisting substantially of, or consisting of at least one extract of each of the following aromatic plants:
aromatic plants of the Lamiaceae family,
aromatic plants of the Asteraceae family, and
aromatic plants of the Poaceae family,
and optionally aromatic plants of the Fabaceae family.

### BRIEF DESCRIPTION OF FIGURE

FIG. 1 is a box diagram (boxplot) showing the comparison of the distributions of the number of fleas per animal (y axis) as a function of the food received (batch A in white or batch B in gray) and as a function of time (x axis; 0=start of the study). The horizontal line in the box indicates the median, the box boundaries indicate the 25th and 75th percentiles, and the whiskers indicate the maximum and minimum results values. The cross (x) inside the box represents the average of the data.

### DETAILED DESCRIPTION

The inventors have surprisingly found that extracts from the aerial parts, roots, seeds or dried fruits of certain aromatic plants described above, or mixtures thereof, have a significant effect in protecting against flea infestation.

The inventors in particular made the surprising finding that the mixture of at least one extract of Lamiaceae, Asteraceae and Poaceae exhibited this effect.

The Lamiaceae are spermaphytic, angiosperm, dicotyledonous and gamopetalous plants of which there are around 200 known species in the French flora. They are herbaceous, annual or perennial plants which are adapted to drought and Mediterranean-type climates. Structurally, these plants have a quadrangular stem, and their leaves are simple, opposite and very variable in shape. Some of the leaves are rolled up from underneath, and are often leathery and thick to better withstand drought.

The Asteraceae family (formerly known as “Compositae”) is an important family of dicotyledonous plants (mainly herbaceous) which includes nearly 13,000 species divided into 1,500 genera. These plants have the common characteristic of having a capitulum inflorescence, i.e. a multitude of stalkless flowers grouped on a receptacle and surrounded by floral bracts. They are found in arid to semi-arid regions in subtropical and lower temperate latitudes.

The Poaceae, or commonly known as grasses, are monocotyledonous angiosperms represented by more than 12,000 species, with a cosmopolitan distribution. They are generally herbaceous plants, more rarely woody, which share morphological characteristics which clearly distinguish them from other plant families: cylindrical stems with hollow internodes, alternate leaves with a distichous arrangement, an elementary inflorescence in spikelets, flowers reduced to sexual organs and fruits of which the pericarp is fused to the seed).

The Fabaceae, or Leguminosae, are a family of dicotyledonous angiosperm plants comprising more than 19,000 species. The Fabaceae, in the broad sense, are herbaceous plants, shrubs, trees or vines. Their distribution is cosmopolitan, and they can be found on all continents, from cold zones to tropical zones.

In the foregoing and what follows, “at least one extract” means one extract or two extracts, or three extracts or more extracts of each of the aromatic plants mentioned.

As such, advantageously, the above-mentioned composition can comprise
two extracts of aromatic plants of the Lamiaceae family,
an extract of aromatic plants of the Asteraceae family, and
an extract of aromatic plants of the Poaceae family,
and optionally an extract of aromatic plants of the Fabaceae family.

Even more advantageously, the above-mentioned composition can comprise
three extracts of aromatic plants of the Lamiaceae family,
an extract of aromatic plants of the Asteraceae family, and
an extract of aromatic plants of the Poaceae family,
and optionally an extract of aromatic plants of the Fabaceae family.

Advantageously, the invention relates to the above-mentioned composition, in which said at least one Lamiaceae extract represents from 5 to 50% by mass, said at least one Asteraceae extract represents from 1 to 20% by mass and said at least one Poaceae extract represents from 1 to 10% by mass, and optionally said at least one Fabaceae extract represents from 5% to 15% by mass,
the percentages being expressed by mass relative to the total mass of the composition.

It is advantageous that the composition according to the invention comprises:
from 5 to 50% by mass of at least one Lamiaceae extract, in particular from 20% to 40% by mass of at least one Lamiaceae extract,
from 1 to 20% by mass of at least one Asteraceae extract, in particular from 5% to 15% by mass of at least one Asteraceae extract, and
from 1% to 10% by mass of at least one Poaceae extract, in particular from 1% to 5% by mass of at least one Poaceae extract,
and optionally from 5% to 15% by mass of at least one Fabaceae extract, in particular from 8% to 12% by mass of at least one Fabaceae extract.

Advantageously, the composition comprises:
from 5 to 50% by mass of two or three Lamiaceae extracts, in particular from 20% to 40% by mass of two or three Lamiaceae extracts,
from 1 to 20% by mass of at least one Asteraceae extract, in particular from 5% to 15% by mass of at least one Asteraceae extract, and
from 1% to 10% by mass of at least one Poaceae extract, in particular from 1% to 5% by mass of at least one Poaceae extract,
and optionally from 5% to 15% by mass of at least one Fabaceae extract, in particular from 8% to 12% by mass of at least one Fabaceae extract.

Advantageously, the invention relates to the above-mentioned composition, comprising, or consisting substantially of, or consisting of a mixture of a lemon balm extract, a thyme extract, a rosemary extract, a wormwood extract and a lemongrass extract, and optionally a fenugreek extract.

Thyme suitable for the present invention, in particular Thymus V, is a plant originating in the Mediterranean basin in the form of a sub-shrub. This plant is generally used for food applications (salad, energy drinks) for both humans and animals, and agricultural applications as a companion plant to protect crops from insects.

Rosemary suitable for the present invention, in particular Rosmarinus O, is a plant originating in the Mediterranean basin in the form of a shrub. This plant is generally used for food applications (salad, energy drinks) for both humans and animals, and agricultural applications as a companion plant to protect crops from insects.

Lemon balm suitable for the present invention, in particular Melissa O, is a plant in the form of a perennial plant. This plant is generally used for food applications (drinks, candy) for both humans and animals, and agricultural applications as a companion plant to protect crops from insects.

Fenugreek, in particular Trigonella Fornum G, is an herbaceous plant characterized by trifoliolate leaves and by a fruit in the form of a pod containing the seeds. This plant is generally used for food applications (drinks, candy) for both humans and animals, and agricultural applications as a companion plant to protect crops from insects.

Wormwood, in particular Artemisia absinthium L., is a species of plant in the Asteraceae family, and is a perennial, herbaceous plant covered with silvery-white silky hairs and numerous oil glands. The stem is silver-green, straight, grooved, branched and very leafy. This plant is generally used for food applications (drinks, candy) for both humans and animals, and agricultural applications as a companion plant to protect crops from insects.

Herbaceous and aromatic, lemongrass, in particular Cymbopogon citratus, is a rhizomatous perennial. It is an excellent companion in the vegetable garden because it keeps leaf miners away. This plant is generally used for food applications (drinks, candy) for both humans and animals, and agricultural applications as a companion plant to protect crops from insects.

More advantageously, the invention relates to the above-mentioned composition, comprising, or consisting substantially of, or consisting of:
from 5% to 50% lemon balm extract,
from 10% to 50% thyme extract,
from 10% to 50% rosemary extract,
from 1% to 20% wormwood extract, and
from 1% to 10% lemongrass extract,
and optionally from 5% to 15% fenugreek extract,
the percentages being expressed by mass relative to the total mass of the composition.

More advantageously, the invention relates to the above-mentioned composition, comprising, or consisting substantially of, or consisting of:
from 15% to 40% lemon balm extract,
from 20% to 40% thyme extract,
from 20% to 40% rosemary extract,
from 5% to 15% wormwood extract, and
from 1% to 5% lemongrass extract,
and optionally from 8% to 12% fenugreek extract,
the percentages being expressed by mass relative to the total mass of the composition.

Other advantageous compositions according to the invention are the following compositions:

### Composition A:

Thyme extract: 40% by mass
Rosemary extract 25% by mass
Lemon balm extract 15% by mass
Fenugreek extract 10% by mass
Wormwood extract 5% by mass
Lemongrass extract 5% by mass
the percentages being expressed by mass relative to the total mass of the composition.

### Composition B:

Thyme extract: 20% by mass
Rosemary extract 40% by mass
Lemon balm extract 20% by mass
Fenugreek extract 10% by mass
Wormwood extract 5% by mass
Lemongrass extract 5% by mass
the percentages being expressed by mass relative to the total mass of the composition.

### Composition C:

Thyme extract: 25% by mass
Rosemary extract 20% by mass
Lemon balm extract 37% by mass
Wormwood extract 15% by mass
Lemongrass extract 1% by mass
the percentages being expressed by mass relative to the total mass of the composition.

Advantageously, the composition according to the invention can also contain vitamins, or amino acids or minerals, or a mixture of thereof.

The invention also relates to a composition for veterinary or pharmaceutical use comprising, or consisting substantially of, or consisting of, as an active substance, the composition as defined above, in association with an acceptable carrier, in particular pharmaceutically or physiologically acceptable.

The composition according to the invention which can be used in food, veterinary or pharmaceutical applications, is advantageously provided for oral administration, and can be formulated in a conventional manner using one or more physiologically acceptable carriers or excipients.

For oral administration, the pharmaceutical or veterinary compositions may be, for example, in the form of tablets, including chewable or suckable tablets, or hard or soft gelatin capsules, including chewable soft gelatin capsules. These compositions can be prepared by conventional methods with pharmaceutically acceptable excipients such as binders (e.g. pre-gelled corn starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); -5-disintegrants (e.g. potato starch or sodium starch glycolate); or wetting agents (e.g. sodium lauryl sulfate).

The tablets can be coated by methods that are well known in the art. Liquid preparations for oral administration can be in the form, for example, of solutions, syrups or suspensions, or they can be presented as a dry product (e.g. in the form of a sachet presentation) for incorporation with water or another suitable carrier before use. Such liquid compositions can be prepared by conventional methods with pharmaceutically acceptable additives such as suspending agents (e.g. sorbitol syrup, cellulose derivatives or hydrogenated edible fats); emulsifying agents (e.g. lecithin or gum arabic); non-aqueous carriers (e.g. almond oil, oily esters, ethyl alcohol or fractionated vegetable oils); and preservatives (e.g. methyl or propyl 2-hydroxybenzoates or sorbic acid).

The preparations may also contain buffering salts, and flavoring, coloring and/or sweetening agents, as appropriate.

Flavoring agents that can be used include, for example, fruit, peppermint or licorice flavorings or palatability agents. The sweetening agents can be, for example, natural sweeteners such as sugars (for example sucrose or fructose) and/or intense sweeteners (for example aspartame, stevia, etc.).

The invention also relates to food intended for feeding non-human animals comprising from 0.01% to 100% of a composition defined above, the percentages being expressed by mass relative to the total mass of the food.

The food according to the invention is either a drink or a solid food.

In the context of the above-mentioned food, the composition according to the invention is advantageously in the form of a solution, an aqueous suspension to be provided in the drinking water or a liquid food, or is in the dry state, adsorbed on a powdered carrier, as well as any other form of supply to the animal food.

The food can consist of the sole composition as defined above, or it can consist of food commonly used in animal nutrition and well known from the prior art (supply of protein, lipids, carbohydrates, minerals and vitamins, etc.) to which from 0.01% to 99.99% of the above composition is added.

In the invention, from 0.01% to 100% corresponds to 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, 10%, 10.1%, 10.2%, 10.3%, 10.4%, 10.5%, 10.6%, 10.7%, 10.8%, 10.9%, 11%, 11.1%, 11.2%, 11.3%, 11.4%, 11.5%, 11.6%, 11.7%, 11.8%, 11.9%, 12%, 12.1%, 12.2%, 12.3%, 12.4%, 12.5%, 12.6%, 12.7%, 12.8%, 12.9%, 13%, 13.1%, 13.2%, 13.3%, 13.4%, 13.5%, 13.6%, 13.7%, 13.8%, 13.9%, 14%, 14.1%, 14.2%, 14.3%, 14.4%, 14.5%, 14.6%, 14.7%, 14.8%, 14.9%, 15%, 15.1%, 15.2%, 15.3%, 15.4%, 15.5%, 15.6%, 15.7%, 15.8%, 15.9%, 16%, 16.1%, 16.2%, 16.3%, 16.4%, 16.5%, 16.6%, 16.7%, 16.8%, 16.9%, 17%, 17.1%, 17.2%, 17.3%, 17.4%1, 17.5%, 17.6%, 17.7%, 17.8%, 17.9%, 18%, 18.1%, 18.2%, 18.3%, 18.4%, 18.5%1, 18.6%, 18.7%, 18.8%, 18.9%, 19%, 19.1%, 19.2%, 19.3%, 19.4%, 19.5%, 19.6%, 19.7%, 19.8%, 19.9%, 20%, 20.1%, 20.2%, 20.3%, 20.4%, 20.5%, 20.6%, 20.7%, 20.8%, 20.9%, 21%, 21.1%, 21.2%, 21.3%, 21.4%, 21.5%, 21.6%, 21.7%, 21.8%, 21.9%, 22%, 22.1%, 22.2%, 22.3%, 22.4%, 22.5%, 22.6%, 22.7%, 22.8%, 22.9%, 23%, 23.1%, 23.2%, 23.3%, 23.4%, 23.5%, 23.6%, 23.7%, 23.8%, 23.9%, 24%, 24.1%, 24.2%, 24.3%, 24.4%, 24.5%, 24.6%, 24.7%, 24.8%, 24.9%, 25%, 25.1%, 25.2%, 25.3%, 25.4%, 25.5%, 25.6%, 25.7%, 25.8%, 25.9%, 26%, 26.1%, 26.2%, 26.3%, 26.4%, 26.5%, 26.6%, 26.7%, 26.8%, 26.9%, 27%, 27.1%, 27.2%, 27.3%, 27.4%, 27.5%, 27.6%, 27.7%, 27.8%, 27.9%, 28%, 28.1%, 28.2%, 28.3%, 28.4%, 28.5%, 28.6%, 28.7%, 28.8%, 28.9%, 29%, 29.1%, 29.2%, 29.3%, 29.4%, 29.5%, 29.6%, 29.7%, 29.8%, 29.9%, 30%, 30.1%, 30.2%, 30.3%, 30.4%, 30.5%, 30.6%, 30.7%, 30.8%, 30.9%, 31%, 31.1%, 31.2%, 31.3%, 31.4%, 31.5%, 31.6%, 31.7%, 31.8%, 31.9%, 32%, 32.1%, 32.2%, 32.3%, 32.4%, 32.5%, 32.6%, 32.7%, 32.8%, 32.9%, 33%, 33.1%, 33.2%, 33.3%, 33.4%, 33.5%, 33.6%, 33.7%, 33.8%, 33.9%, 34%, 34.1%, 34.2%, 34.3%, 34.4%, 34.5%, 34.6%, 34.7%, 34.8%, 34.9%, 35%, 35.1%, 35.2%, 35.3% 35.4%, 35.5%, 35.6%, 35.7%, 35.8%, 35.9%, 36%, 36.1%, 36.2%, 36.3%, 36.4%, 36.5%, 36.6%, 36.7%, 36.8%, 36.9%, 37%, 37.1%, 37.2%, 37.3%, 37.4%, 37.5% 37.6%, 37.7%, 37.8%, 37.9%, 38%, 38.1%, 38.2%, 38.3%, 38.4%, 38.5%, 38.6%, 38.7%, 38.8%, 38.9%, 39%, 39.1%, 39.2%, 39.3%, 39.4%, 39.5%, 39.6%, 39.7% 39.8%, 39.9%, 40%, 40.1%, 40.2%, 40.3%, 40.4%, 40.5%, 40.6%, 40.7%, 40.8%, 40.9%, 41%, 41.1%, 41.2%, 41.3%, 41.4%, 41.5%, 41.6%, 41.7%, 41.8%, 41.9%, 42%, 42.1%, 42.2%, 42.3%, 42.4%, 42.5%, 42.6%, 42.7%, 42.8%, 42.9%, 43%, 43.1%, 43.2%, 43.3%, 43.4%, 43.5%, 43.6%, 43.7%, 43.8%, 43.9%, 44%, 44.1%, 44.2%, 44.3%, 44.4%, 44.5%, 44.6%, 44.7%, 44.8%, 44.9%, 45%, 45.1%, 45.2%, 45.3%, 45.4%, 45.5%, 45.6%, 45.7%, 45.8%, 45.9%, 46%, 46.1%, 46.2%, 46.3%, 46.4%, 46.5%, 46.6%, 46.7%, 46.8%, 46.9%, 47%, 47.1%, 47.2%, 47.3%, 47.4% 47.5%, 47.6%, 47.7%, 47.8%, 47.9%, 48%, 48.1%, 48.2%, 48.3%, 48.4%, 48.5%, 48.6%, 48.7%, 48.8%, 48.9%, 49%, 49.1%, 49.2%, 49.3%, 49.4%, 49.5%, 49.6%, 49.7%, 49.8%, 49.9%, 50%, 50.1%, 50.2%, 50.3%, 50.4%, 50.5%, 50.6%, 50.7%, 50.8%, 50.9%, 51%, 51.1%, 51.2%, 51.3%, 51.4%, 51.5%, 51.6%, 51.7%, 51.8%, 51.9%, 52%, 52.1%, 52.2%, 52.3%, 52.4%, 52.5%, 52.6%, 52.7%, 52.8%, 52.9%, 53%, 53.1%, 53.2%, 53.3%, 53.4%, 53.5%, 53.6%, 53.7%, 53.8%, 53.9%, 54%, 54.1%, 54.2%, 54.3%, 54.4%, 54.5%, 54.6%, 54.7%, 54.8%, 54.9%, 55%, 55.1%, 55.2%, 55.3%, 55.4%, 55.5%, 55.6%, 55.7% 55.8%, 55.9% 56%, 56.1%, 56.2%, 56.3%, 56.4%, 56.5%, 56.6%, 56.7%, 56.8%, 56.9%, 57%, 57.1%, 57.2%, 57.3% 57.4%, 57.5%, 57.6%, 57.7%, 57.8%, 57.9%, 58%, 58.1%, 58.2%, 58.3%, 58.4%, 58.5%, 58.6%, 58.7%, 58.8%, 58.9%, 59%, 59.1%, 59.2%, 59.3%, 59.4%, 59.5% 59.6%, 59.7%, 59.8%, 59.9%, 60%, 60.1%, 60.2%, 60.3%, 60.4%, 60.5%, 60.6%, 60.7%, 60.8%, 60.9%, 61%, 61.1%, 61.2%, 61.3%, 61.4%, 61.5%, 61.6%, 61.7%, 61.8%, 61.9%, 62%, 62.1%, 62.2%, 62.3%, 62.4%, 62.5%, 62.6%, 62.7%, 62.8%, 62.9%, 63%, 63.1%, 63.2%, 63.3%, 63.4%, 63.5%, 63.6%, 63.7%, 63.8%, 63.9%, 64%, 64.1%, 64.2%, 64.3%, 64.4%, 64.5%, 64.6%, 64.7%, 64.8%, 64.9%, 65%, 65.1%, 65.2%, 65.3%, 65.4%, 65.5%, 65.6%, 65.7%, 65.8%, 65.9%, 66%, 66.1%, 66.2%, 66.3%, 66.4%, 66.5%, 66.6%, 66.7%, 66.8%, 66.9%, 67%, 67.1%, 67.2%, 67.3%, 67.4%, 67.5%, 67.6%, 67.7%, 67.8%, 67.9%, 68%, 68.1%, 68.2%, 68.3%, 68.4%, 68.5%, 68.6%, 68.7%, 68.8%, 68.9%, 69%, 69.1%, 69.2%, 69.3%, 69.4%, 69.5%, 69.6%, 69.7%, 69.8%, 69.9%, 70%, 70.1%, 70.2%, 70.3%, 70.4%, 70.5%, 70.6%, 70.7%, 70.8%, 70.9%, 71%, 71.1%, 71.2%, 71.3%, 71.4%, 71.5%, 71.6%, 71.7%, 71.8%, 71.9%, 72%, 72.1%, 72.2%, 72.3%, 72.4%, 72.5%, 72.6%, 72.7%, 72.8%, 72.9%, 73%, 73.1%, 73.2%, 73.3%, 73.4%, 73.5%, 73.6%, 73.7%, 73.8%, 73.9%, 74%, 74.1%, 74.2%, 74.3%, 74.4%, 74.5%, 74.6%, 74.7%, 74.8%, 74.9% 75%, 75.1%, 75.2%, 75.3%, 75.4%, 75.5%, 75.6%, 75.7%, 75.8%, 75.9%, 76%, 76.1%, 76.2%, 76.3%, 76.4%, 76.5%, 76.6%, 76.7%, 76.8%, 76.9%, 77%, 77.1%, 77.2%, 77.3%, 77.4%, 77.5%, 77.6%, 77.7%, 77.8%, 77.9%, 78%, 78.1%, 78.2%, 78.3%, 78.4%, 78.5%, 78.6%, 78.7%, 78.8%, 78.9%, 79%, 79.1%, 79.2%, 79.3% 79.4%, 79.5%, 79.6%, 79.7%, 79.8%, 79.9%, 80%, 80.1%, 80.2%, 80.3%, 80.4%, 80.5%, 80.6%, 80.7%, 80.8%, 80.9%, 81%, 81.1%, 81.2%, 81.3%, 81.4%, 81.5%, 81.6%, 81.7%, 81.8%, 81.9%, 82%, 82.1%, 82.2%, 82.3%, 82.4%, 82.5%, 82.6%, 82.7%, 82.8%, 82.9%, 83%, 83.1%, 83.2%, 83.3%, 83.4%, 83.5%, 83.6%, 83.7%, 83.8%, 83.9%, 84%, 84.1%, 84.2%, 84.3%, 84.4%, 84.5%, 84.6%, 84.7%, 84.8%, 84.9%, 85%, 85.1%, 85.2%, 85.3%, 85.4%, 85.5%, 85.6%, 85.7%, 85.8%, 85.9%, 86%, 86.1%, 86.2%, 86.3%, 86.4%, 86.5%, 86.6%, 86.7%, 86.8%, 86.9%, 87%, 87.1%, 87.2%, 87.3%, 87.4%, 87.5%, 87.6%, 87.7%, 87.8%, 87.9%, 88%, 88.1%, 88.2%, 88.3%, 88.4%, 88.5%, 88.6%, 88.7%, 88.8%, 88.9%, 89%, 89.1%, 89.2%, 89.3%, 89.4%, 89.5%, 89.6%, 89.7%, 89.8%, 89.9%, 90%, 90.1%, 90.2%, 90.3%, 90.4%, 90.5%, 90.6%, 90.7%, 90.8%, 90.9%, 91%, 91.1%, 91.2%, 91.3%, 91.4%, 91.5%, 91.6%, 91.7%, 91.8%, 91.9%, 92%, 92.1%, 92.2%, 92.3%, 92.4%, 92.5%, 92.6%, 92.7%, 92.8%, 92.9%, 93%, 93.1%, 93.2%, 93.3%, 93.4%, 93.5%, 93.6%, 93.7%, 93.8%, 93.9%, 94%, 94.1%, 94.2%, 94.3%, 94.4%, 94.5%, 94.6%, 94.7% 94.8%, 94.9%, 95%, 95.1%, 95.2%, 95.3%, 95.4%, 95.5%, 95.6%, 95.7%, 95.8%, 95.9%, 96%, 96.1%, 96.2%, 96.3%, 96.4%, 96.5%, 96.6%, 96.7%, 96.8%, 96.9%, 97%, 97.1%, 97.2%, 97.3%, 97.4%, 97.5%, 97.6%, 97.7%, 97.8%, 97.9%, 98%, 98.1%, 98.2%, 98.3%, 98.4%, 98.5%, 98.6%, 98.7%, 98.8%, 98.9%, 99%, 99.1%, 99.2%, 99.3%, 99.4%, 99.5%, 99.6%, 99.7%, 99.8%, 99.9%, 99.91%, 99.92%, 99.93%, 99.94%, 99.95%, 99.96%, 99.97%, 99.98%, 99.99% or 100%.

The invention also relates to the above-mentioned composition as a drug, or for use as a drug, in particular for veterinary use.

The invention further relates to the above-mentioned composition as a food supplement, flavoring agent or other product intended for food.

In the context of a drug for veterinary use, it is advantageous to have a composition in liquid form to be used in pure form or to be added to drinking water (for dilution), or in a dry state adsorbed on a food carrier, in particular a powdered carrier.

The food supplement is advantageously administered, or is in a form suitable for administration, throughout the life of the animal, of the order of 1 ml to 20 ml per animal, in particular once a day.

The invention also relates to the above-mentioned composition for use in the treatment of domestic mammals for the prevention or treatment of flea infestation (also known as pulicosis), and the prevention of the associated symptoms.

Domestic mammals according to the invention are pet mammals which can be infected by fleas, and in particular without being limiting: cats and dogs, hedgehogs and rabbits. The composition according to the invention is particularly advantageous for dogs and cats.

As shown in the examples below, the composition according to the invention makes it possible to limit infestation by fleas, and therefore to prevent infestation. It also helps prevent complications and symptoms linked to the presence of fleas, such as pruritus, erythema, scales, secondary injuries (excoriations, scabs, etc.), allergic dermatitis, and infections by microorganisms such as lichenification or infections by bacteria responsible for cat scratch disease, or infections by digestive worms Dipylidium caninum, or tapeworms.

The invention further relates to a method for preparing the above-mentioned composition, in which:
lemon balm, thyme, rosemary, wormwood and lemongrass leaves are compacted in a sufficient volume of water to cover them, to obtain a mixture of compacted leaves,
said mixture of compacted leaves is left to macerate for at least two weeks at room temperature, with regular mixing of said mixture of compacted leaves,
the macerate is eliminated to preserve the liquid fraction,
and, optionally, the liquid fraction is mechanically stirred to obtain a homogeneous composition.

The composition according to the invention is therefore obtained by a simple inexpensive method, which does not involve any chemical agent.

The composition is obtained in liquid form as a result of the above-mentioned method and, as mentioned above, can be used directly in pure liquid form or added to drinking water, or can be mixed with food, for example kibbles, either by impregnation of the kibbles or by adding the composition adsorbed on a powdered carrier.

The invention will be better understood in the light of the FIGURE and the following examples:

### Examples

### Example 1: Method for preparing a composition according to the invention

The method for producing the food supplement comprises the steps of:
Introducing water into a tank. The volume of the mixture corresponds to one third of the volume of the tank.
Introducing the leaves of the aromatic plants, starting with the lowest quantity.
Compacting the plants with a spatula.
If necessary, adding a water mixture to completely cover the plants.
Covering the tank with the cover and leaving it to macerate at room temperature (15 to 27° C.) for at least 2 weeks, while stirring the mixture regularly with a spatula, in particular every 3 days.
Filtering the mixture by clarifying filtration through a sieve.
Optionally adding additional active ingredients (minerals, vitamins, extracts, etc.).
Stirring the extract using a mechanical stirrer set at approx. 100 rpm for 12 hours to obtain a homogeneous composition.

The composition thus obtained is optionally sprayed into a mixer containing a powdered food carrier (before being packaged, to obtain a food comprising the composition according to the invention.

The food is thus ready to be distributed to the animals.

Finally, the constituents of the composition can be sterilized, in particular by heating and/or by passing through a membrane. They can be disinfected, also by heating or by means of a disinfectant.

### Example 2: Measurement of the Effectiveness of the Composition According to the Invention

### 1—Purpose of the Study

The aim of this study was to evaluate, in a double-blind, placebo-controlled setting, the effectiveness of the composition according to the invention used alone, in the summer, to limit flea populations in naturally infested dogs living in dirt-floor kennels without environmental treatment.

### 2—Material and Method

### 2.1 Animals.

Twenty-two healthy adult dogs aged 1 to 11 years were included, and they were housed in 7 semi-open kennels in groups of 1 to 5. Their food consisted exclusively of the food provided by the study sponsor, and was distinguishable only by the letter on the outer packaging (A or B). There was plenty of water available. Throughout the study, accommodation and food conditions remained stable. No anti-parasitic drugs were allowed. No external anti-parasitic treatment had been administered during the past 6 months. The description of each animal appears in Table 1.

TABLE 1
animal report

| No. | | | | | | |
|---|---|---|---|---|---|---|
| in the | | Age | | | Weight | |
| study | Sex | (years) | Breed | Kennel | (kg) | Group |
| 1 | M | 8 | Bruno Jura Hound | 1 | 27 | A |
| 2 | M | 3 | Griffon | 1 | 24 | A |
| 3 | F | 11 | Bruno Jura Hound | 1 | 25 | A |
| 4 | F | 11 | Bruno Jura Hound | 1 | 27 | A |
| 10 | F | 6 | Bleu de Gascogne | 3 | 24 | A |
| 11 | M | 1 | Griffon | 3 | 18 | A |
| 12 | F | 2 | Bleu de Gascogne | 3 | 20 | A |
| 13 | F | 3 | Griffon | 3 | 19 | A |
| 19 | M | 3 | Griffon | 6 | 25 | A |
| 20 | M | 7 | Griffon | 6 | 35 | A |
| 21 | M | 3 | Griffon | 6 | 23 | A |
| 22 | M | 7 | Griffon | 7 | 30 | A |
| 5 | M | 6 | Bruno Jura Hound | 2 | 27 | B |
| 6 | M | 5 | Bleu de Gascogne | 2 | 25 | B |
| 7 | M | 1 | Griffon | 2 | 20 | B |
| 8 | M | 3 | Griffon | 2 | 31 | B |
| 9 | M | 1 | Griffon | 2 | 22 | B |
| 14 | M | 5 | Griffon | 4 | 23 | B |
| 15 | M | 7 | Griffon | 4 | 30 | B |
| 16 | M | 1 | Bleu de Gascogne | 4 | 22 | B |
| 17 | M | 2 | Bleu de Gascogne | 4 | 22 | B |
| 18 | M | 5 | Griffon | 5 | 31 | B |

### 2.2 the Product.

The active ingredient to be evaluated was incorporated into the food before sending it to the animal keeper. The bags (12 kg each) could only be distinguished by the letter “A” or “B” on the packaging. The bags were stored in a suitable dry place at a temperature below 25° C.

The food consists of dehydrated chicken meat (43%), dehydrated whole potato, potato pulp, peas, poultry fat, dehydrated shrimp (4%), animal protein hydrolysate, kaolin (2%), minerals, gluten-free flax meal, dehydrated Yucca schidigera extract, dehydrated brewer's yeast, dehydrated chicory pulp, and a premix of flavoring additives with active substances from citrus fruits. This food is constituted by mass relative to the total mass of the food: 34% crude protein, 12% crude fat, 3% crude fiber, 6% mineral material, 0.75% calcium, 0.47% phosphorus, 0.20% omega 3 and 1.10% omega 6, with a Ca/P ratio of 1.55.

The food was distributed once a day by the owner, and the quantity distributed was based on the manufacturer's recommendations for animals with intense activity of more than two hours of exercise so as to adequately cover the needs. Within the same pen, all dogs were fed the same food.

### 2.3 Method

### 2.3.1 Evaluation of the Anti-Parasitic Activity.

Counting was carried out according to the method recommended by the European Medicines Agency (EMEA) for carnivores in field trials (Guideline for the testing and evaluation of the efficacy of antiparasitic substances for the treatment and prevention of tick and flea infestation in dogs and cats EMEA CVMP EWP 005 2000—Rev. 3-14 Jul. 2016). The flea population of each dog is assessed by visual counting. Area counts are performed on five body regions of each animal: dorsal line, base of tail, right side, left side and inguinal region. For each area, the counting time is limited to one minute and the counting is carried out by parting the hairs using both hands until the surface of the area is covered. For each area, the maximum number of fleas counted is limited to 50, which brings the maximum number of fleas evaluated per animal to 250.

The counting was carried out before the start of the study (DO), then 14 days, and 1, 2, 3, 4 and 5 months after the start of the supplementation.

### 2.3.2 Clinical Evaluation

### 2.3.2.1 Tolerance.

Immediate tolerance was assessed at the time of each meal by the owner. At each follow-up visit, local and general clinical examination was used to assess medium-term tolerance.

### 2.3.2.2 Dermatological Monitoring

The evaluation took place before the start of the study (DO), then 14 days, and 1, 2, 3, 4 and 5 months after the start of the supplementation. For each animal, the intensity of the following injuries was graded on a scale of 0 to 3 [0: absence; 1: moderate presence; 2: average presence; 3: significant presence]:
infected area
pruritus intensity
erythema
scales
secondary injuries (excoriations, scabs, etc.)
lichenification

The clinical score corresponds to the sum of the evaluations per injury.

### 2.4 Analysis of the Results

The flea counts of the two groups were compared by a test t for two independent samples with a significance level of 5%. The calculations were performed using the XLSTAT software (Addinsoft, v. 19.02 43358).

### 3 Results

### 3.1 Tolerance

No intolerance reaction was detected after distribution of the food. In post-application clinical examinations, no clinical abnormalities were detected.

Dog 6 was excluded from the study as of D60, having been moved out of the kennel.

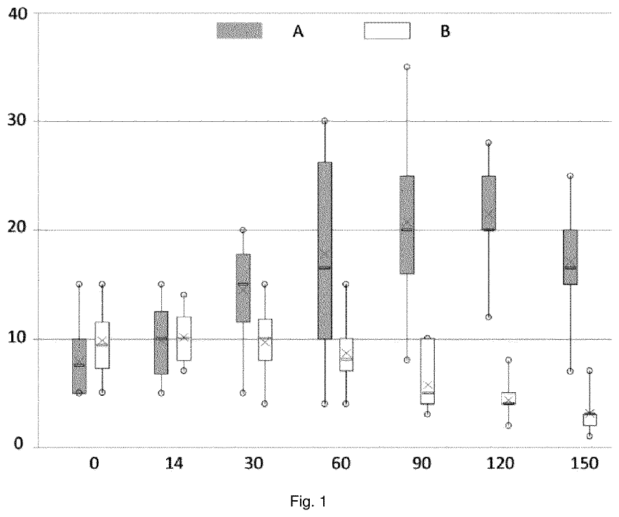

### 3.2 Anti-Parasitic Efficacy (Table 2 and FIG. 1).

On inclusion in the study, the dogs were harboring between 5 and 15 fleas, i.e. on average 7.9±3.3 fleas for batch A (12 dogs) and 9.5±3.6 fleas for batch B (10 dogs).

The results of the study are shown in FIG. 1, and in Table 2 below.

| | | 30-Jul | 14-Aug | 28-Aug | 25-Sep | 26-Oct | 20-Nov | 18-Dec |
|---|---|---|---|---|---|---|---|---|
| Batch | Dog | D0 | D14 | D30 | D60 | D90 | D120 | D150 |
| A | 1 | 5 | 5 | 20 | 25 | 25 | 25 | 20 |
| | 2 | 8 | 12 | 20 | 30 | 35 | 28 | 25 |
| | 3 | 5 | 7 | 10 | 12 | 25 | 20 | 15 |
| | 4 | 7 | 10 | 10 | 10 | 13 | 15 | 12 |
| | 10 | 5 | 7 | 15 | 10 | 19 | 20 | 15 |
| | 11 | 10 | 14 | 20 | 30 | 12 | 25 | 20 |
| | 12 | 15 | 14 | 15 | 15 | 17 | 20 | 15 |
| | 13 | 10 | 12 | 12 | 10 | 20 | 20 | 16 |
| | 19 | 5 | 6 | 5 | 4 | 8 | 12 | 7 |
| | 20 | 5 | 6 | 15 | 20 | 30 | 28 | 25 |
| | 21 | 12 | 15 | 17 | 18 | 25 | 20 | 17 |
| | 22 | 8 | 10 | 15 | 30 | 20 | 25 | 18 |
| B | 5 | 10 | 12 | 12 | 15 | 4 | 4 | 4 |
| | 6 | 5 | 7 | 8 | — | — | — | — |
| | 7 | 5 | 8 | 4 | 8 | 10 | 5 | 3 |
| | 8 | 8 | 7 | 15 | 6 | 10 | 5 | 3 |
| | 9 | 8 | 9 | 8 | 7 | 5 | 4 | 3 |
| | 14 | 15 | 13 | 10 | 4 | 3 | 2 | 2 |
| | 15 | 15 | 12 | 12 | 12 | 10 | 8 | 7 |
| | 16 | 12 | 14 | 11 | 10 | 4 | 4 | 2 |
| | 17 | 7 | 8 | 7 | 7 | 5 | 4 | 3 |
| | 18 | 10 | 11 | 10 | 10 | 3 | 3 | 1 |
| Avg | A | 7.9 | 9.8 | 14.5 | 17.8 | 20.8 | 21.5 | 17.1 |
| Avg | B | 9.5 | 10.1 | 9.7 | 8.8 | 6.0 | 4.3 | 3.1 |
| SD | A | 3.3 | 3.6 | 4.6 | 9.1 | 7.7 | 4.9 | 5.1 |
| SD | B | 3.6 | 2.6 | 3.1 | 3.3 | 3.1 | 1.7 | 1.7 |

| | | | | | | |
|---|---|---|---|---|---|---|
| Statistics | NS | S | S | S | S | S |
| % reduction B/A | / | 33.1% | 50.8% | 71.1% | 79.8% | 81.8% |

The average flea population hosted by the dogs of group B remained stable during the first month of the study and then gradually decreased to 3.1±1.7 at month 5. At the same time and under the same rearing and maintenance conditions, the average population of fleas in group A increased steadily until month 4 (21.5±4.9) and then decreased significantly in month 5 (17.1±5.1). The reduction percentages of batch B compared to batch A are 33%, 51%, 71%, 80% and 82%, respectively, at one, two, three, four and five months after the start of the study.

Statistical analysis showed no significant difference on DO. The averages of the two groups are significantly different between D14 and D0+5 months (p<0.05).

### 3.3 Skin Examination

Skin examinations generally revealed good skin and coat quality, apart from scales noted at 1/3 for some dogs at the start and during the study:
D0: dogs 12, 14, 15 and 21
D14: dogs 12, 14, 15 and 21
D30: dogs 12, 14, 15 and 21
D60: dogs 2, 12, 15 and 21
D90: dogs 2, 12, 15, 20 and 21
D120: dogs 2, 12, 20 and 21
D150: dogs 2, 12, 20 and 21

### 4 Conclusion

Flea populations in naturally infested dogs, with an average level of infestation at the start of the study (8 to 9.5 fleas on average) and living in dirt-floor kennels without environmental treatment were monitored for 5 months. In the batch provided with food A, the populations increased until they reached an average of greater than 20 fleas per animal; they were significantly higher than those of the dogs provided with food B, which gradually decreased to reach averages of less than 5 fleas per dog. The intake of the food did not cause any adverse event.

At the end of the study, the veterinarian was informed of the composition of food B, which contained the following composition according to the invention:
Lemon balm extract: 37%
Thyme extract: 25%
Rosemary extract: 20%
Wormwood extract: 15%
Lemongrass extract: 1%
the percentages being expressed by mass relative to the total mass of the composition, and the composition being used at a rate of 1% by mass relative to the mass of the food to which it is added to feed the animals.

1. A method for the prevention of flea infestation (pulicosis) in a domestic mammal, the method comprising: administering to the domestic mammal an effective amount of a composition consisting of one of the following mixtures:
a) a mixture of a lemon balm extract, a thyme extract, a rosemary extract, a wormwood extract and a lemongrass extract; or
b) a mixture of a lemon balm extract, a thyme extract, a rosemary extract, a wormwood extract, a lemongrass extract and a fenugreek extract.

2. The method of claim 1, wherein the composition is defined by the following:
if the composition is mixture a), wherein:
the lemon balm extract represents from 5% to 50%,
the thyme extract represents from 10% to 50%,
the rosemary extract represents from 10% to 50%,
the wormwood extract represents from 1% to 20%, and
the lemongrass extract represents from 1% to 10%; or
if the composition is mixture b), wherein:
the lemon balm extract represents from 5% to 50%,
the thyme extract represents from 10% to 50%,
the rosemary extract represents from 10% to 50%,
the wormwood extract represents from 1% to 20%,
the lemongrass extract represents from 1% to 10%, and
the fenugreek extract represents from 5% to 15%.
the percentages being expressed by mass relative to the total mass of the composition.

3. The method of claim 1, wherein the composition is mixture a) and wherein:
the lemon balm extract represents from 15% to 40%,
the thyme extract represents from 20% to 40%,
the rosemary extract represents from 20% to 40%,
the wormwood extract represents from 5% to 15%, and
the lemongrass extract represents from 1% to 5%.

4. The method of claim 3, wherein the composition consists of:
37% by mass of lemon balm extract,
25% by mass of thyme extract,
20% by mass of rosemary extract,
15% by mass of wormwood extract, and
1% by mass of lemongrass extract.

5. The method of claim 1 wherein the composition further comprises vitamins, or amino acids or minerals, or a mixture thereof.

6. The method of claim 1, wherein the domestic mammal is a dog or a cat.

7. The method of claim 1, wherein the composition is administered orally.

8. The method of claim 1, wherein the composition is administered by incorporation into a food product.

9. A method for the treatment of flea infestation (pulicosis) in a domestic mammal, the method comprising: administering to the domestic mammal an effective amount of a composition consisting of one of the following mixtures:
a) a mixture of a lemon balm extract, a thyme extract, a rosemary extract, a wormwood extract and a lemongrass extract; or
b) a mixture of a lemon balm extract, a thyme extract, a rosemary extract, a wormwood extract, a lemongrass extract and a fenugreek extract.

10. The method of claim 9, wherein the composition is defined by the following:
if the composition is mixture a), wherein:
the lemon balm extract represents from 5% to 50%,
the thyme extract represents from 10% to 50%,
the rosemary extract represents from 10% to 50%,
the wormwood extract represents from 1% to 20%, and
the lemongrass extract represents from 1% to 10%; or
if the composition is mixture b), wherein:
the lemon balm extract represents from 5% to 50%,
the thyme extract represents from 10% to 50%,
the rosemary extract represents from 10% to 50%,
the wormwood extract represents from 1% to 20%,
the lemongrass extract represents from 1% to 10%, and
the fenugreek extract represents from 5% to 15%.
the percentages being expressed by mass relative to the total mass of the composition.

11. The method of claim 9, wherein the composition is mixture a) and wherein:
the lemon balm extract represents from 15% to 40%,
the thyme extract represents from 20% to 40%,
the rosemary extract represents from 20% to 40%,
the wormwood extract represents from 5% to 15%, and
the lemongrass extract represents from 1% to 5%.

12. The method of claim 11, wherein the composition consists of:
37% by mass of lemon balm extract,
25% by mass of thyme extract,
20% by mass of rosemary extract,
15% by mass of wormwood extract, and
1% by mass of lemongrass extract.

13. The method of claim 9 wherein the composition further comprises vitamins, or amino acids or minerals, or a mixture thereof.

14. The method of claim 9, wherein the domestic mammal is a dog or a cat.

15. The method of claim 9, wherein the composition is administered orally.

16. The method of claim 9, wherein the composition is administered by incorporation into a food product.
